(19) United States
(12) Patent Application Publication
(10) Pub. No.: US 2015/0305635 A1
(43) Pub. Date: Oct. 29, 2015

(54) PORTABLE ELECTRONIC DEVICE AND HEART RATE SENSING METHOD OF THE SAME

(71) Applicant: EMINENT ELECTRONIC TECHNOLOGY CORP. LTD., Hsinchu (TW)

(72) Inventors: Tom Chang, Taipei City (TW); Kao-Pin Wu, New Taipei City (TW); Chih-Jen Fang, Tainan City (TW); Yuan-Shun Yeh, Zhubei City (TW); Wei-Te Hsu, New Taipei City (TW); De-Cheng Pan, Lukang Township (TW)

(21) Appl. No.: 14/688,169

(22) Filed: Apr. 16, 2015

Related U.S. Application Data
(60) Provisional application No. 61/983,470, filed on Apr. 24, 2014.

(30) Foreign Application Priority Data
Feb. 10, 2015 (TW) 104104308

(51) Int. Cl. A61B 5/024; A61B 5/00

(52) U.S. Cl. CPC A61B 5/02438; A61B 5/6898; A61B 5/02427

(57) ABSTRACT

A portable electronic device uses the initial heartbeat value to calculate a possible heartbeat variation. Then a reference range is determined. Only the real time heartbeat value falling in the reference range is output as a correct real time heartbeat value. Thus, the too large or too small erroneous heartbeat values influenced by the vibration noise are eliminated to precisely output the correct real time heartbeat value. Therefore, the extra vibration sensors need not to be installed to lower the cost and to reduce the volume.

### CROSS-REFERENCE TO RELATED APPLICATIONS

This application claims the benefit of United States provisional application filed on Apr. 24, 2014 and having application Ser. No. 61/983,470, the entire contents of which are hereby incorporated herein by reference

This application is based upon and claims priority under 35 U.S.C. 119 from Taiwan Patent Application No. 104104308 filed on Feb. 10, 2015, which is hereby specifically incorporated herein by this reference thereto.

### BACKGROUND OF THE INVENTION

1. Field of the Invention

The present invention relates to a portable electronic device and a heartbeat value sensing method of the same, especially to an electronic device detects blood by light detectors and its sensing method.

2. Description of the Prior Arts

With the progress of the technology, the portable electronic device can provide various functions. One of the application of the portable electronic device is to detect heartbeat value. The user puts the finger or other part of the body on a light source. A light detector receives the reflecting light of the light source to generate a blood detecting signal. A control unit calculates the heartbeat value based on the blood detecting signal. Thus, the user can measure his heartbeat value at rest, or monitor his heartbeat value during exercise.

However, the user's body is unavoidably vibrated during exercise. When detecting heartbeat value during exercise, the blood detecting signal generated by the light detector includes a real signal from the real heartbeat value and a vibration signal from the vibration so that the heartbeat value calculated by the blood detecting signal may be incorrect. The conventional portable electronic device has an extra gravity sensor (G-sensor) to detect the vibration signal. Then the control unit can eliminate the vibration signal influence to calculate a correct heartbeat value. However, mounting extra G-sensor not only increases manufacturing cost but also enlarges the volume so that is not unfavorable for the portable electronic device to be lightweight, especially to wearable devices. The overweight or oversized wearable devices are undoubtedly less valued.

To overcome the shortcomings, the present invention provides a portable electronic device and a heartbeat value sensing method of the same to mitigate or obviate the aforementioned problems.

### SUMMARY OF THE INVENTION

The present invention seeks solution to obtain a correct heartbeat value that is not influenced by the vibration noise without having extra components.

To achieve the aforementioned objectives, the present invention provides a portable electronic device comprising:

a first light source providing a first beam;

a light detector comprising at least one light detecting unit, wherein the light detector detects a reflected light generated from the first beam of the first light source emitting to a user's body, and then a blood detecting signal is generated based one the reflected light;

a control unit connecting to the first light source and the light detector and executing a real time heartbeat value sensing method having following steps:

(a) obtaining an initial heartbeat value;

(b) setting a reference range based on the initial heartbeat value;

(c) obtaining a real time heartbeat value; and

(d) determining whether the real time heartbeat value falls into the reference range; when the real time heartbeat value falls into the reference range, the real time heartbeat value is output; when the real time heartbeat value does not fall into the reference range, the real time heartbeat value is not output.

The advantage of the present invention is to use the initial heartbeat value to set the reference range so that the unusual values occurred by the vibration noise is eliminated without mounting extra components. Then the user's heartbeat value is calculated accurately. Therefore, the manufacturing cost and the volume of the portable electronic device are reduced and the portable electronic device is easily worn or carried by the user.

Other objectives, advantages and novel features of the invention will become more apparent from the following detailed description when taken in conjunction with the accompanying drawings.

### BRIEF DESCRIPTION OF THE DRAWINGS

FIG. 1 is an illustrated view of a portable electronic device in accordance with the present invention;

FIG. 2 is a block diagram of the portable electronic device in FIG. 1;

FIG. 3 is a block diagram of the circuit of the light detector of the portable electronic device in FIG. 1;

FIG. 4 is a flow chart of a heartbeat value sensing method of the portable electronic device in accordance with the present invention;

FIG. 5 is a flow chart of obtaining heartbeat value of the method in FIG. 4;

FIG. 6 is a flow chart of a proximity sensing procedure of the method in FIG. 4; and

FIG. 7 is an illustrative view of another embodiment of a portable electronic device in accordance with the present invention.

### DETAILED DESCRIPTION OF THE PREFERRED EMBODIMENTS

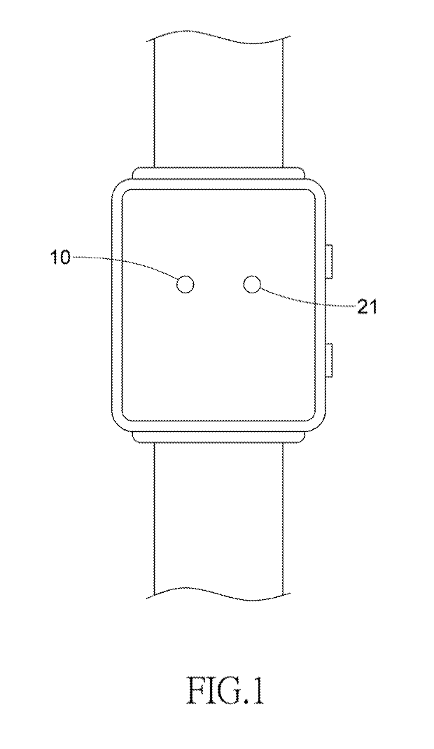

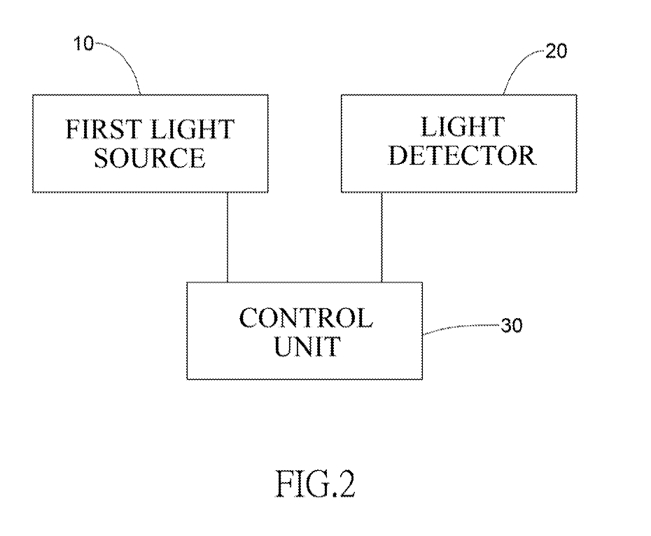

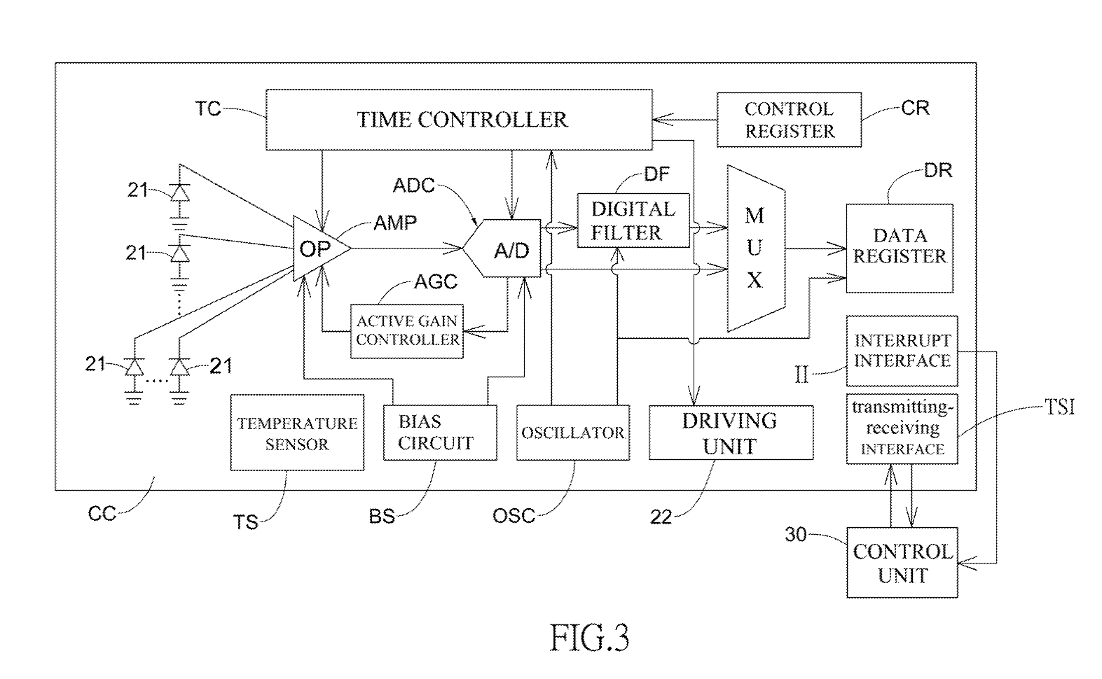

With reference to FIGS. 1 to 3, a portable electronic device in accordance with the present invention comprises a first light source 10, a light detector 20 and a control unit 30.

The light detector 20 has at least one light detecting unit 21 and at least one driving unit 22. Each driving unit 22 is connected to and drives the first light source 10. The light detecting unit 21 receives the lights through holes on the portable electronic device as described to generate light detecting signal. The light source 10 emits light through the holes on the portable electronic device as described. The amount and the relating locations of the light detecting units 21 and the driving units 22 are regarded as the functions provided by the portable electronic device as described. The related location of the first light source 10 and the light detecting unit 21 is also regarded as the functions provided by the portable electronic device as described. Further, the light detecting units 21 and the light source 10 may be integrated in a semiconductor package. In a preferred embodiment, the light detector 20 comprises multiple light detecting units 21, a time controller TC, an amplifier AMP, an active gain controller AGC, an analog-to-digital converter ADC, a digital filter DF, a multiplexer MUX, a control register CR, a data register DR, an interrupt interface II, a transmitting-receiving interface TSI, a light source controller LC, an oscillator OSC, a bias circuit BC and a temperature sensor TS. The light detecting unit 21 detects light to generate said light detecting signal. The amplifier AMP amplifies the light detecting signal and the amplification of the amplifier AMP is adjusted by the active gain controller AGC. The active gain controller AGC adjusts the amplification of the amplifier AMP and the integration time of the light detecting unit 21 for the light detecting signal to reach the desired brightness. The analog-to-digital converter ADC converts the amplified light detecting signal to a digital signal. The digital filter DF filters noises. The time controller TC controls the time sequences of the elements in the light detector 20. The temperature sensor TS detects the temperature. The bias circuit BS is a bias voltage source of the analog circuit. The oscillator OSC provides the clock signal. The light source controller LC controls the said light sources 30. The control register CR and the data register DR respectively storage commands and detecting results. The transmitting-receiving interface TSI transmits and receives the commands and the data. The interrupt interface II notifies the control unit 30 about the condition of the storage space to determine the data whether transmitted or received.

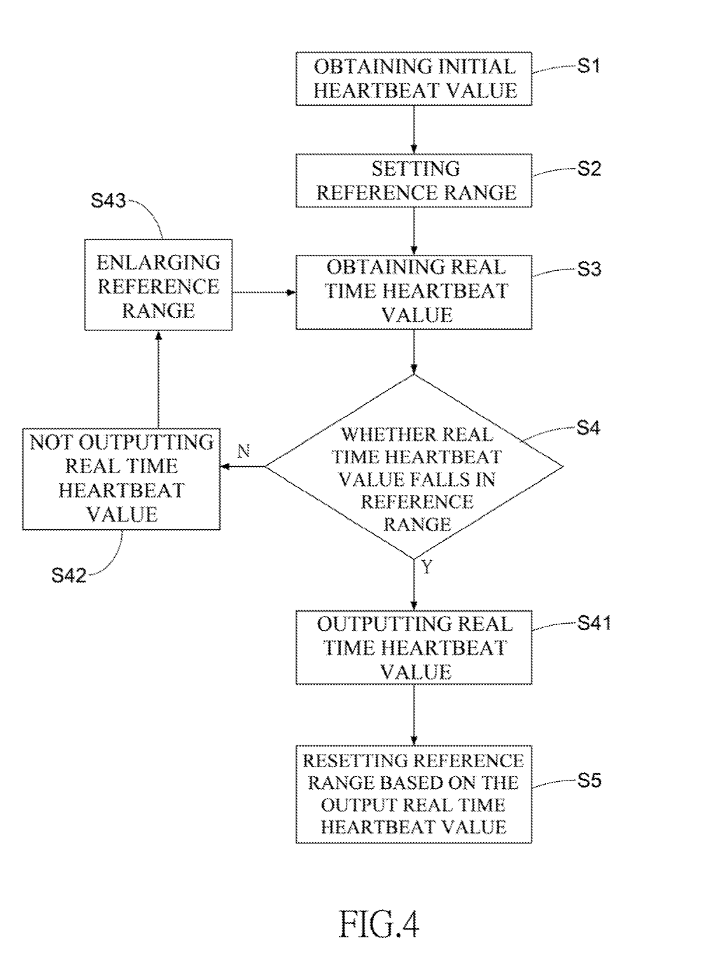

With reference to FIGS. 1, 2 and 4, a heartbeat value sensing method in accordance with the present invention executing by the control unit 30 comprises following steps:

Obtaining initial heartbeat value (S1): When the user's body approaches the first light source 10, the first light source 10 emits a first beam to the user's body to generate a reflected light. The light detecting unit 21 of the light detector 20 receives the reflected light and generates a blood detecting signal to obtain the initial heartbeat value.

Setting a reference range (S2): The initial heartbeat value is used to set up a reference range of a possible heartbeat value variation. In one embodiment, the initial heartbeat value is used as a median value to set the reference range. For example, the initial heartbeat value is 72 times per minute. Suppose the heartbeat value cannot change more than 30 times in difference. Thus, the reference range can be set as 42 to 102 times per minute or even smaller range such as 52 to 92 times per minute.

Obtaining a real time heartbeat value (S3): The first light source 10 emits the first beam to the user's body again to generate a reflected light. The light detecting unit 21 of the light detector 20 receives the reflected light and generates a blood detecting signal to obtain the real time heartbeat value.

Determining whether the real time heartbeat value falling in the reference range (S4): The obtained real time heartbeat value is compared with the reference range to determine whether the real time heartbeat value falling in the reference range. Whether the real time heartbeat value does fall in the reference range, the real time heartbeat value is output (S41). Whether the real time heartbeat value does fall in the reference range, the real time heartbeat value is not output (S42). Then the reference range is enlarged (S43) and then go back to step S3. In step S43 that enlarging the reference range, the reference range is slightly enlarged as predetermined, but the enlarged range does not exceed a reasonable value that predetermined by the system so that the result influenced by the vibration noise is not determined as the real time heartbeat value.

Therefore, with the comparison between the reference range and the real time heartbeat value, the output real time heartbeat value is always a correct heartbeat value that falls in the reasonable variation range and is not an incorrect heartbeat value influenced by the vibration noise.

Further, the method as described further comprises another step of resetting the reference range (S5): The output real time heartbeat value is used to reset the reference range. In one embodiment, the real time is used as a median value to reset the reference range. With the user changes his condition, the real time heartbeat value may be changed as well. For example, when the user exercises, the real time heartbeat value definitely changed. Therefore, the reference range is adjusted by the real time heartbeat value to accurately determine whether the next real time heartbeat value is correct.

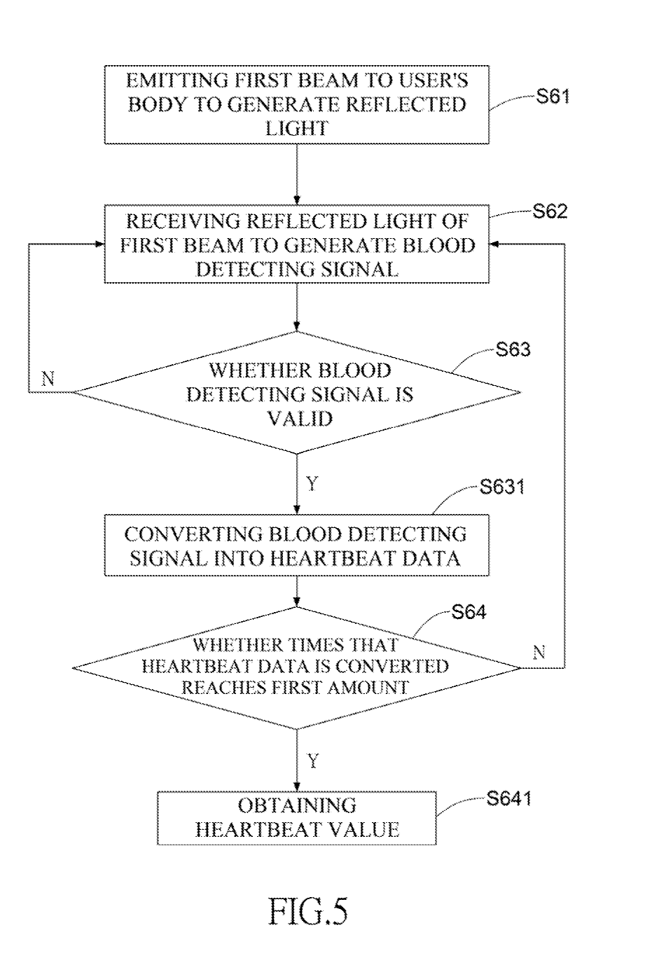

Moreover, with further reference to FIG. 5, the steps S1 and S3 may comprises following sub-steps:

Emitting the first beam to the user's body (S61): The first light source 10 emits a first beam to the user's body to generate a reflected light.

Receiving the reflected light of the first beam (S62): The light detecting unit 21 of the light detector 20 receives the reflected light and generates a blood detecting signal.

Determining whether the blood detecting signal is valid (S63): The control unit 30 determines whether the blood detecting signal is valid. The blood detecting signal may be invalid because the user's body moves away or other reason to result in the blood detecting signal being too small. The invalid blood detecting signal is unable to be used to further determine the heartbeat value. Whether the blood detecting signal is determined as valid, the blood detecting signal is converted into a heartbeat data (S631). Whether the blood detecting signal is determined as valid, then go back to step S62.

Determining whether the times that the heartbeat data is converted reaches a certain amount (S64): When the blood detecting signal is converted into the heartbeat data, the times that the heartbeat data is converted are calculated to determine whether the times reach the certain amount, such as 100 to 500 times. Whether the times reach the certain amount, a heartbeat value is calculated by the all converted heartbeat data. Whether the times are not reached the certain amount yet, then go back to step S62.

By determining whether the blood detecting signal is valid, a threshold may be set to eliminate the over-large and over-small signal. With multiple heartbeat data to obtain the heartbeat value, a single event occurred by a surge is kept from influencing the heartbeat value so that the calculated heartbeat value is more accurate.

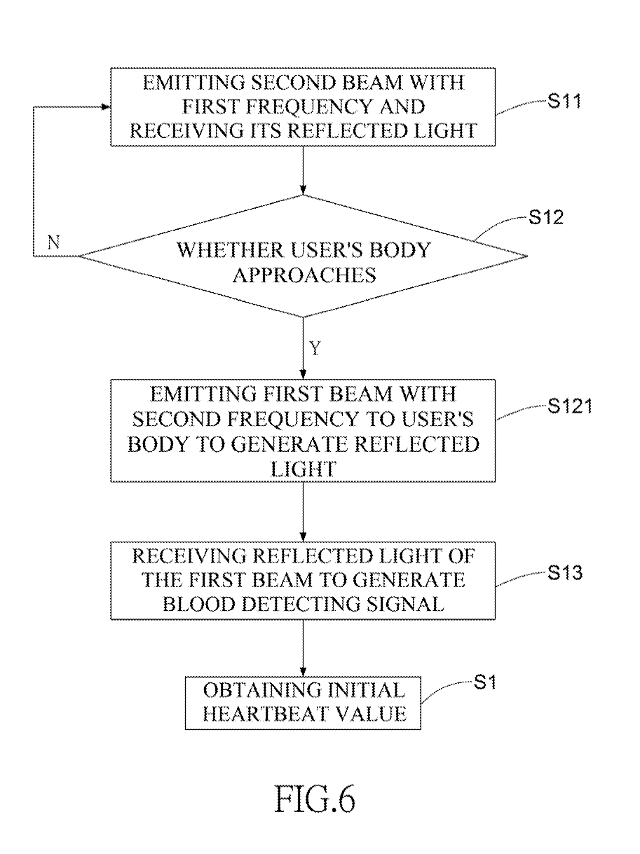

With further reference to FIG. 6, a following proximity sensing procedure is executed before executed step S1 to save power:

Emitting a second beam with a first emitting frequency and receiving its reflected light (S11): The first light source 10 emits the second beam with the first emitting frequency. Then the light detecting unit 21 of the light detector 20 receives the reflected light of the second beam.

Determining whether a user's body approaches (S12): The received reflected light is used to determine whether the user's body approaches close enough. When the user's body approaches closer, the intensity of the reflected light is stronger. When the user's body is determined close enough, the first beam is emitted to the user's body with a second emitting frequency to generate a reflected light (S121). When the user's body is determined not close enough or no user's body approach is determined, go back to step S11.

Receiving the reflected light of the first beam (S13): The light detecting unit 21 of the light detector 20 receives the reflected light and generates a blood detecting signal.

Obtaining initial heartbeat value (S1): The blood detecting signal is used to obtain the initial heartbeat value.

With the aforementioned proximity sensing procedure, the second beam is emitted with the lower first emitting frequency when the portable electronic device is idle. When the user's body approach is determined, the first beam is emitted with the higher second emitting frequency to obtain the initial heartbeat value. The lower frequency cost lower power so that using the proximity sensing procedure can reduce power consumption.

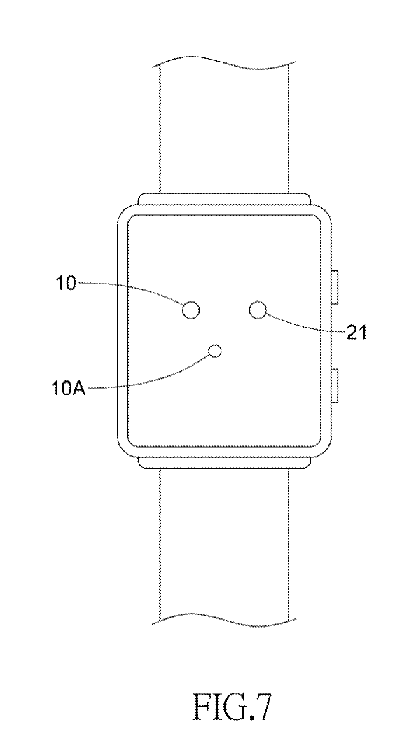

Further, with reference to FIG. 7, the portable electronic device further comprises a second light source 10A. The second light source 10A emits the second light with the first emitting frequency. In one embodiment, the second light source 10A emits invisible light so that the user's vision is not influenced by the second beam when the portable electronic device is idle.

Moreover, when the initial heartbeat value is obtained, the initial heartbeat value is converted into an initial frequency. Then the initial frequency is used to set up the reference range. When the real time heartbeat value is obtained, the real time heartbeat value is converted into a real time frequency to be compared with the initial frequency.

Therefore, the portable electronic device as described uses the initial heartbeat value to set the reference range to compare with the subsequent real time heartbeat values. Thus, when the false heartbeat value that is too large or too small due to the vibration noise, the false heartbeat value is eliminated because of the comparison result between the false heartbeat value and the reference range. Then the output real time heartbeat value is accurate and is not influenced by the vibration noise.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A sensing method for a real time heartbeat value comprising steps of:
(a) obtaining an initial heartbeat value;
(b) setting a reference range based on the initial heartbeat value;
(c) obtaining a real time heartbeat value; and
(d) determining whether the real time heartbeat value falls into the reference range; when the real time heartbeat value falls into the reference range, the real time heartbeat value is output; when the real time heartbeat value does not fall into the reference range, the real time heartbeat value is not output.

2. The sensing method as claimed in claim 1 further comprising a following step after executing the step (d):
(e) resetting the reference range based on the real time heartbeat value when the real time heartbeat value is determined as falling in the reference range.

3. The sensing method as claimed in claim 1, wherein the step (a) comprises following sub-steps:
(a1) emitting a first beam to a user's body to generate a reflected light;
(a2) receiving the reflected light of the first beam to generate a blood detecting signal;
(a3) obtaining the initial heartbeat value based on the blood detecting signal.

4. The sensing method as claimed in claim 3 further comprising a following step after executing the step (a2):
(a21) determining whether the blood detecting signal is valid; executing the step (a2) when the blood detecting signal is invalid; executing the step (a3) when the blood detecting signal is valid.

5. The sensing method as claimed in claim 3, further comprising a following step after executing the step (a2):
(a21) determining whether the blood detecting signal is valid; executing the step (a2) when the blood detecting signal is invalid;
(a22) converting the blood detecting signal as a heartbeat data when the blood detecting signal is valid;
(a23) determining whether the times that the heartbeat data is converted reaches a first amount; executing the step (a2) when the times that the heartbeat data is converted does not reach the first amount; and
(a24) executing the step (a3) when the times that the heartbeat data is converted reaches the first amount.

6. The sensing method as claimed in claim 1, wherein the step (c) comprises following sub-steps:
(c1) emitting a first beam to a user's body to generate a reflected light;
(c2) receiving the reflected light of the first beam to generate a blood detecting signal;
(c3) obtaining the real time heartbeat value based on the blood detecting signal.

7. The sensing method as claimed in claim 6 further comprising a following step after executing the step (c2):
(c21) determining whether the blood detecting signal is valid; executing the step (c2) when the blood detecting signal is invalid; executing the step (c3) when the blood detecting signal is valid.

8. The sensing method as claimed in claim 6, further comprising a following step after executing the step (c2):
(c21) determining whether the blood detecting signal is valid; executing the step (c2) when the blood detecting signal is invalid;
(c22) converting the blood detecting signal as a heartbeat data when the blood detecting signal is valid;
(c23) determining whether the times that the heartbeat data is converted reaches a second amount; executing the step (c2) when the times that the heartbeat data is converted does not reach the second amount; and
(c24) executing the step (c3) when the times that the heartbeat data is converted reaches the second amount.

9. The sensing method as claimed in claim 1 further executing following steps before executing the step (a):
(a01) emitting a second beam with a first emitting frequency and receiving a reflected light of the second beam to determine whether a user's body approaches close enough to a position where the reflected light is received; executing the step (a02) when the user's body is determined close enough; repeating the step (a01) when no user's body is determined close enough;
(a02) emitting a first beam with a second emitting frequency to the user's body to generate a reflected light, wherein the second emitting frequency is larger than the first emitting frequency; and
(a03) receiving the reflected light of the first beam to generate a blood detecting signal.

10. The sensing method as claimed in claim 9, wherein the first beam and the second beam are emitted from the same light source.

11. The sensing method as claimed in claim 9, wherein the first beam and the second beam are emitted from different light sources and the second beam is invisible beam.

12. The sensing method as claimed in claim 1, wherein
in the step (a), an initial frequency is obtained based on the initial heartbeat value;
in the step (b), the reference range is set by using the initial frequency as a median value;
in the step (c), a real time frequency is obtained based on the real time heartbeat value; and
in the step (d), whether real time frequency falls into the reference range is determined.

13. The sensing method as claimed in claim 2, wherein
in the step (a), an initial frequency is obtained based on the initial heartbeat value;
in the step (b), the reference range is set by using the initial frequency as a median value;
in the step (c), a real time frequency is obtained based on the real time heartbeat value;
in the step (d), whether real time frequency falls into the reference range is determined; and
in the step (e), the reference range by using the real time frequency as a median value is reset when the real time frequency falls into the reference range.

14. The sensing method as claimed in claim 1, wherein in the step (d), the reference range is enlarged and then the step (c) is executed when the real time heartbeat value is not output.

15. A portable electronic device comprising:
a first light source providing a first beam;
a light detector comprising at least one light detecting unit, wherein the light detector detects a reflected light generated from the first beam of the first light source emitting to a user's body, and then a blood detecting signal is generated based one the reflected light;
a control unit connecting to the first light source and the light detector and executing following steps:
(a) obtaining an initial heartbeat value;
(b) setting a reference range based on the initial heartbeat value;
(c) obtaining a real time heartbeat value; and
(d) determining whether the real time heartbeat value falls into the reference range; when the real time heartbeat value falls into the reference range, the real time heartbeat value is output; when the real time heartbeat value does not fall into the reference range, the real time heartbeat value is not output.

16. The portable electronic device as claimed in claim 15, wherein the control unit further executes a following step after executes the step (d):
(e) resetting the reference range based on the real time heartbeat value when the real time heartbeat value is determined as falling in the reference range.

17. The portable electronic device as claimed in claim 15, wherein the control unit executes the step (a) comprising following sub-steps:
(a1) driving the first light source to emit the first beam to the user's body to generate the reflected light;
(a2) controlling the light detector to receive the reflected light of the first beam to generate the blood detecting signal;
(a3) obtaining the initial heartbeat value based on the blood detecting signal.

18. The portable electronic device as claimed in claim 17, wherein the control unit further executes a following step after executes the step (a2):
(a21) determining whether the blood detecting signal is valid; executing the step (a2) when the blood detecting signal is invalid; executing the step (a3) when the blood detecting signal is valid.

19. The portable electronic device as claimed in claim 17, wherein the control unit further executes a following step after executes the step (a2):
(a21) determining whether the blood detecting signal is valid; executing the step (a2) when the blood detecting signal is invalid;
(a22) converting the blood detecting signal as a heartbeat data when the blood detecting signal is valid;
(a23) determining whether the times that the heartbeat data is converted reaches a first amount; executing the step (a2) when the times that the heartbeat data is converted does not reach the first amount; and
(a24) executing the step (a3) when the times that the heartbeat data is converted reaches the first amount.

20. The portable electronic device as claimed in claim 15, wherein the control unit executes the step (c) comprising following sub-steps:
(c1) driving the first light source to emit the first beam to the user's body to generate the reflected light;
(c2) controlling the light detector to receive the reflected light of the first beam to generate the blood detecting signal;
(c3) obtaining the real time heartbeat value based on the blood detecting signal.

21. The portable electronic device as claimed in claim 20 wherein the control unit further executes a following step after executes the step (c2):
(c21) determining whether the blood detecting signal is valid; executing the step (c2) when the blood detecting signal is invalid; executing the step (c3) when the blood detecting signal is valid.

22. The portable electronic device as claimed in claim 20, wherein the control unit further executes a following step after executes the step (c2):
(c21) determining whether the blood detecting signal is valid; executing the step (c2) when the blood detecting signal is invalid;
(c22) converting the blood detecting signal as a heartbeat data when the blood detecting signal is valid;
(c23) determining whether the times that the heartbeat data is converted reaches a second amount; executing the step (c2) when the times that the heartbeat data is converted does not reach the second amount; and
(c24) executing the step (c3) when the times that the heartbeat data is converted reaches the second amount.

23. The portable electronic device as claimed in claim 15 wherein the control unit further executes following steps before executes the step (a):
(a01) driving the first light source to emit a second beam with a first emitting frequency and receiving a reflected light of the second beam to determine whether the user's body approaches close enough to the light detector; executing the step (a02) when the user's body is determined close enough; repeating the step (a01) when no user's body is determined close enough;
(a02) driving the first light source to emit the first beam with a second emitting frequency to the user's body to generate the reflected light, wherein the second emitting frequency is larger than the first emitting frequency; and
(a03) controlling the light detector to receive the reflected light of the first beam to generate the blood detecting signal.

24. The portable electronic device as claimed in claim 15 further comprising a second light source connecting to the control unit, wherein the control unit further executes following steps before executes the step (a):
(a01) driving the second light source to emit a second beam with a first emitting frequency and receiving a reflected light of the second beam to determine whether the user's body approaches close enough to the light detector; executing the step (a02) when the user's body is determined close enough; repeating the step (a01) when no user's body is determined close enough;
(a02) driving the first light source to emit the first beam with a second emitting frequency to the user's body to generate the reflected light, wherein the second emitting frequency is larger than the first emitting frequency; and
(a03) controlling the light detector to receive the reflected light of the first beam to generate the blood detecting signal.

25. The portable electronic device as claimed in claim 24, wherein the second beam is invisible beam.

26. The portable electronic device as claimed in claim 15, wherein:
when the control unit executes the step (a), an initial frequency is obtained based on the initial heartbeat value;
when the control unit executes the step (b), the reference range is set by using the initial frequency as a median value;
when the control unit executes the step (c), a real time frequency is obtained based on the real time heartbeat value; and
when the control unit executes the step (d), whether real time frequency falls into the reference range is determined.

27. The portable electronic device as claimed in claim 16, wherein:
when the control unit executes the step (a), an initial frequency is obtained based on the initial heartbeat value;
when the control unit executes the step (b), the reference range is set by using the initial frequency as a median value;
when the control unit executes the step (c), a real time frequency is obtained based on the real time heartbeat value;
when the control unit executes the step (d), whether real time frequency falls into the reference range is determined; and
when the control unit executes the step (e), the reference range by using the real time frequency as a median value is reset when the real time frequency falls into the reference range.

28. The portable electronic device as claimed in claim 15, wherein when the control unit executes the step (d), the reference range is enlarged and then the step (c) is executed when the real time heartbeat value is not output.
